(19) United States
(12) Patent Application Publication
(10) Pub. No.: US 2007/0005072 A1
(43) Pub. Date: Jan. 4, 2007

(54) Pedicle punch

(75) Inventors: Hector Castillo, Mastic, NY (US); Stefano Sinicropi, Bronx, NY (US)

Correspondence Address: Thomas A. O'Rourke, Esq.;Bodner & O' Rourke, LLP, Suite 108, 425 Broadhollow Road, Melville, NY, 11747, US

(73) Assignee: ORTHO IMPACT INC.

(21) Appl. No.: 11/156,100

(22) Filed: Jun. 18, 2005

(51) Int. Cl. A61B 17/00

(52) U.S. Cl. 606/79

(57) ABSTRACT

A pedicle punch used to establish pilot holes in vertebra pedicles when deployed thereon during pedicle screw insertion processes comprising a proximal end, a distal end, a shaft, a lip, a radiation opaque center core, and a radiation translucent outer layer. The proximal end acts as a handle for the pedicle punch. The shaft distally projects from the center of the proximal end and terminates to a sharp conical spike at the distal end. The lip outwardly projects from and is fixedly connected to the shaft, adjacent to the sharp conical spike and concentric thereto. The radiation opaque center core runs longitudinally along the entire length of the pedicle punch, through the proximal end, through the shaft and lip outwardly projecting therefrom, to the very tip of the distal end; and the radiation translucent outer layer concentrically surrounds and encases the radiation opaque center core, running along the entire length of the pedicle punch. However, it stops short a short distance from the very tip of the distal end to allow the radiation opaque center core to extend sufficiently beyond the radiation translucent outer layer and form the bottom of the sharp conical spike.

### BACKGROUND OF THE INVENTION

1. Field of the Invention

The present invention relates to a method and apparatus for use in spinal implant procedures. More particular, it relates to a new and improved pedicle punch for use in accurately placing and forming pilot holes on the pedicles of vertebrae, said pilot holes having a diameter sufficiently wide to accommodate further instrumentation, and an initial trajectory path vector concentric to and aligned with the pedicle axis, whereby the penetration of the pedicle with a pedicle probe and the subsequent insertion into the pedicle of a pedicle screw is accomplished quickly and accurately, and without breaking out of the pedicle during a spinal fusion operation.

2. Related and Prior Art Statement

Instances arise when it becomes necessary to stabilize or fuse a portion of the spine from motion such as, for example (1) after decompression wherein certain posterior spinal elements are removed to relieve pressure or neural elements, (2) after trauma, or (3) because of the presence of tumors. Instrument systems that accomplish spinal fixation generally comprise pedicle screws which are adapted to be inserted in selected vertebrae, and stiff rods or plates that connect adjacent pedicle screw heads to one another after the screws are inserted, thus resulting in the fixing or bracing of all vertebrae spanned by the rod or plate. Commercially available pedicle screws are usually made of stainless steel having overall diameters (including threads) generally ranging from 4.5 mm and 7.5 mm and lengths that vary depending on the instrumentation.

The pedicle of a vertebra is a dense, stem-like structure projecting from the posterior of the vertebra. There are two pedicles per vertebra that connect to other structures. The pedicle is the strongest point of attachment of the spine. FIG. 1 herein is a diagram illustrating a vertebra with the pedicle shown from all angles.

Since the pedicles are the strongest parts of the spinal vertebrae, they provide a secure foundation for the pedicle screws to which fixing rods or plates are attached. In order to derive the greatest mechanical integrity when anchoring pedicle screws in a spine-fixing instrument system, it is essential that the screws be guided and threaded in alignment with the pedicle axis and not be allowed to deviate off axis, in which case the screw body or its threads will break through the vertebral cortex and impinge on, or become dangerously close to, surrounding nerve roots. The deeper the screws are inserted in the pedicle, the more stable vertebral bodies will be fixed. Consequently, deviations in the angle of screw insertion can injure nerve roots and the spinal cord and lead to vascular injury.

FIG. 2 herein shows a pedicle conceptualized as a cylinder. If the proposed pedicle screw trajectory enters at the top of this cylinder (the pedicle screw entry site) at or medial to its lateral wall and exits the bottom of the cylinder (the junction of the pedicle and the vertebral body) lateral to its medial wall (enough to accommodate the anticipated screw diameter), the pedicle can be safely navigated.

Generally speaking, procedures for navigating the pedicle comprise the following steps: (1) decorticating the entry site using a burr and a high speed drill or a rongeur; (2) using an awl or a burr to penetrate the dorsal cortex of the pedicle and create a starter or pilot hole into the pedicle; (3) using a curved or straight pedicle probe to develop a path for the screw through the cancellous bone of the pedicle into the vertebral body (the process hereinafter referred to as “cannulation”). The advancement of the probe must be smooth and consistent and a sudden plunge suggests breaking out of the pedicle laterally. Furthermore, an increase in resistance indicates abutment against the pedicle or the vertebral body; (4) after cannulation, placing the pedicle sounding probe into the pedicle and then palpating the pedicle from within to make sure there is not a medial, lateral, rostral or caudal disruption in the cortex of the pedicle. Sound should also be used to determine that there is bone at the bottom of the pilot hole verifying that penetration of the ventral cortex of the vertebral body has not occurred; (5) after the pedicles have been probed, placing Steinman pins or K-wires bilaterally or unilaterally into the pedicles to confirm the trajectory and entry site, tapping the pedicle screw path if non-self tapping screws are used, and placing the permanent screws with the longest diameter that will not fracture the pedicle. The length of the screw can be determined by measuring the length of the Steinman pin/K-wire/pedicle probe from the pedicle entry site to a depth of 50-80% of the vertebral body; and (6) after pedicle screw placement, decorticating the transverse process and the lateral aspects of the facet joints, connecting the screw to a longitudinal construct, usually a rod or a plate, securing the screws, and placing bone graf on the previously fusion bed. During the entire process the advancement of the probe, the placement of metallic wires, and the ultimate advancement of the pedicle screws can be monitored with varying amounts of X-ray exposure or fluoroscopy.

These processes have many disadvantages including, among other things, a steep learning curve, caudal or medial penetration of the pedicle cortex which can result in dural or neural injury, and a very lengthy operative time with potential or significant blood loss, and an increased risk of infection. Furthermore, these processes only approximate or simulate screw placement indirectly, through a CT scan or MRI done prior to surgery and include fluoroscopic and frameless stereotactic guidance.

The overuse of X-rays and fluoroscopy during these processes is also a drawback. The reality is that radiation is a hazard. Ionizing radiation has no safe, threshold of exposure below which it ceases to have adverse effects, although an arbitrary level is assumed. There has been a recent upward revision of risk estimates of radiation exposure but absolute levels of safe exposure remain unknown. Exposure to the surgical team as well as the patient during the spinal fusion process using fluoroscopy is a universal concern. Yet notwithstanding the use of all of this radiation, much appears in the literature with respect to the problems of misalignment of pedicle screws and the symptoms arising when the screws make contact with neural elements after breaking outside the pedicle cortex. Cutting into a nerve root or simply contacting the root gives rise to various postoperative symptoms, including dropped foot, neurological lesions, sensory deficits or pain.

The relevant and material prior art has tried to address some of these drawbacks. One example of improvements in the prior art that have tried to address these disadvantages is U.S. Pat. No. 6,855,105 B2. It is directed to a system and method for insertion of pedicle screws which eliminates the guesswork and error-prone modalities of the prior art and provides the surgeon with direct confirmation during the surgical procedure that the pedicle probe is in the right position for forming a path for the proper placement of the pedicle screw. The system and method comprises an endoscopic pedicle probe for use during spinal surgery to form a path in a pedicle for reception of a pedicle screw. The probe has an enlarged proximal end for cooperation with the hand of the surgeon so that the probe can be pushed through the pedicle in a controlled manner, and an elongated hollow shaft terminating in a distal tip end. A fiber optic cable or endoscope is placed in the hollow shaft and connected with a monitor to enable the surgeon to visually observe the structure adjacent the tip end of the probe during surgery, whereby the probe may be accurately placed in the pedicle for subsequent accurate placement of the pedicle screw in the path formed with the probe.

Another example, is U.S. Pat. No. 5,573,537. It is directed to a combination reaming and probing instrument and a method for its use to install pedicle screws. The instrument provides a step-down screw hole in a pedicle. It includes an end member for rotatable connection with a power source, a probe for providing a pilot hole in the pedicle and a side-cutting reamer member for providing an anchor hole for a pedicle screw positioned about the pilot hole. The arrangement is such that the instrument is self-centering in part due to the presence of the probe in the pilot hole.

A further example is U.S. Pat. No. 5,361,766 for a quick release bone probe and x-ray marker for use in spinal implant procedures to enter the pedicle of a vertebrae. It comprises a probe having a proximal end and a distal end with fastening means on the proximal end and a handle. The handle has gripping means and further includes fastening means for selectively engaging the proximal end of the probe. The distal end of the probe is shaped to pierce the pedicle of a vertebrae. The probe includes marking means for determining the depth the distal end projects into a pedicle and marking means for selectively distinguishing a right probe from a left probe on interoperative x-rays.

Yet another example is U.S. Pat. No. 4,790,297 which is directed to a spinal fixation method and system which allows direct screw fixation to the vertebral bodies to produce the potential for a very stable fusion. Blunt guide wires inserted into the pedicles help preclude penetration of the anterior cortex while permitting more accurate screw placement with the assistance of X-ray imaging.

Still another example in the prior art is U.S. Pat. No. 5,196,015 which is directed to a procedure for spinal pedicle screw insertion in spinal vertebrae in a manner to reduce the likelihood of nerve damage caused by improper screw placement. It comprises the following steps: a screw opening is started in part of a skeletal region, e.g., a pedicle of a lumbar vertebra and an electric potential of a certain magnitude is applied to the inner surface of the opening while the patient is observed for nervous reactions such as leg twitching. The opening continues to be formed while the electric potential is applied until a desired hole depth is obtained in the absence of nervous reaction to the potential. The direction in which the screw opening is being formed is changed to a direction other than the last direction, after observing patient reactions to the electric potential when the screw opening was being formed in the last direction. A tool for carrying out the procedure includes a handle and a probe tip extending from the handle for forming an opening in bone tissue. Stimulator circuitry arranged inside the handle produces an electric potential of a predetermined level, and the potential is applied to the probe tip while the tool handle is manipulated to urge the probe tip into the bone tissue.

Another example is U.S. Pat. No. 4,907,577 directed to a spinal transpedicle drill which is adapted for providing a safe route for drilling, including an I-shaped body, a guiding base and a positioning base. The jig provides a precise location for drilling to prevent deviation of the drilling direction so as to prevent injury during surgery to the nerve root or spinal cord.

Yet another example is U.S. Pat. No. 4,586,497 directed to a drill fixation device and method for vertebral cutting. The device includes a carrier mounting the drill for guided movement relative to a base and a squeeze grip connected between the base and carrier to enable a surgeon to selectively impart such movement to the drill. A foot plate is fixed relative to the base in spaced relationship to the carrier so as to be in the path of the bit of a drill mounted on the carrier. In use, the foot plate is positioned beneath the bone material to be cut to apply counter force to the bone material as the drill cuts and shield nerve tissue from harm by the bit.

However, all of the improvements set forth above and many others, both patented and unpatented, focus on changing and improving traditionally accepted practices and procedures for pedicle screw insertion, after the initial steps of using a burr or an awl to penetrate and create starter holes, entry ports, or pilot holes in the dorsal cortex of the vertebrae pedicle, have taken place. Thus, these improvements have done nothing to change the steep learning curve of the surgeons in defining, marking and creating the pilot holes during the pedicle screw insertion process, or to significantly reduce the very high subsequent misplacement rate of pedicle screws. While pedicle disruption does not necessarily cause neural deficit, keeping the pedicle probe and ultimately the pedicle screw within the pedicle is one sure way to prevent it altogether. Further, these improvements do absolutely nothing to significantly reduce the amount of radiation, the amount of time it takes to complete the pedicle insertion and the steps necessary for it.

As was discussed above, procedures for navigating the pedicle comprise the initial steps of decorticating the entry site using a burr and a high speed drill or a rongeur; and using an awl or a burr, with the aid of X-rays or fluoroscopy, to penetrate the dorsal cortex of the pedicle and create a starter or pilot hole into the pedicle Other procedures include burning to first mark the starting point and then burring to create the pilot hole on the pedicle. There are significant drawbacks with these steps as well.

If the initial pilot holes on the pedicles are accomplished through burning, such markings are impermanent. Further, they do not create the necessary 3-4 mm starting hole required for the next instrumentation tools, i.e. the probe and ultimately the pedicle screws. Consequently, burning must be followed by burring or using an awl to create the pilot holes. This in turn increases the number of steps in the pedicle screw insertion process and thereby the time necessary to complete the process. Furthermore, use of an awl to create a pilot hole, particularly in the thoracic spine area, is inherently dangerous since an awl has nothing to prevent the surgeon from plunging it into the spinal cord.

Furthermore, if burring or an awl are used to create all the starting or pilot holes, it is very difficult to create them without moving the x-ray or fluoroscopic image while utilizing the burr and other instrumentation. In addition, the burring of starting or pilot holes can cause significant bleeding thereby requiring the administration of thrombotic agents during the procedure to stop the blood flow. This bleeding can be further aggravated if, as a result of false burring starts, a large number of starting but useless holes are created. Finally, because fluoroscopy has to frequently be repeated before, during and after the starting or pilot hole is created due to the difficulty associated with burring using fluoroscopy, the method often requires two or three attempts with image guidance, thus significantly increasing the time it takes to complete the pedicle screw insertion. More importantly however, even with the two or three attempts with image guidance, the fluoroscopy image does shift during the burning or burring, thereby making it impossible to set a pilot hole trajectory path whose vector is aligned with the pedicle axis right from the very beginning of the pedicle penetration process. Consequently, when the surgeon proceeds to the next step of inserting the probe or any other instrument he will use to enlarge the pedicle path, in preparation of the insertion of the pedicle screw, he must correct the initial vector of the pilot hole trajectory path and compensate for any errors or deviations thereof from the pedicle axis, if the insertion of the pedicle screw is to proceed smoothly and with out pedicle break through. This only adds to the time it takes to complete the process and increases the radiation used during the process even further.

Accordingly, there is a need for a pedicle screw insertion system and method which creates a pedicle starter hole having a trajectory path vector in as complete alignment with the pedicle axis as possible, right from the very beginning of the process, thereby minimizing any need on the part of the surgeon to correct or compensate for any deviations of the pilot hole's trajectory path vector from the pedicle axis during the subsequent steps of the process, and nearly eliminates the creation of erroneous, multiple pilot holes on the vertebrae pedicle.

### OBJECTS OF THE INVENTION

IT IS THEREFORE AN OBJECT of the present invention to provide a method and apparatus for use in spinal implant procedures wherein the penetration of the pedicle with a probe and subsequent insertion of the pedicle screw is accomplished quickly, and accurately and without breaking out of the pedicle path.

IT IS A FURTHER OBJECT of the present invention to provide a method and apparatus for use in spinal implant procedures that will significantly decrease the surgeons' learning curve in placing pilot holes on the vertebrae pedicles, reduce the caudal or medial penetration of the pedicle cortex thereby significantly reducing dural or neural injury, minimize the time normally associated with such procedures, and practically eliminate the blood loss and the risk for infection due to improper pilot hole placement.

IT IS AN EVEN FURTHER OBJECT of the present invention to provide a method and apparatus for use in spinal implant procedures that will significantly reduce the exposure of both the surgical team and the patient to radiation exposure and more particularly to fluoroscopic radiation exposure.

IT IS STILL ANOTHER OBJECT of the present invention to provide a method and apparatus for use in spinal implant procedures which may significantly minimize the administration of thrombotic agents during the pedicle insertion process.

ANOTHER OBJECT of the present invention is to provide a method and apparatus for use in spinal implant procedures which will almost eliminate the creation of false burring starts and significantly reducing the number of starting or pilot holes.

IT IS A FURTHER OBJECT of the present invention to provide a method and apparatus for use in spinal implant procedures in which the starter hole or pilot hole has a trajectory path whose vector is in complete alignment with the pedicle axis right from the very beginning of the pedicle penetration process.

IT IS YET ANOTHER OBJECT of the present invention to provide a method and apparatus for use in a spinal implant procedure wherein the surgeon's need to correct the initial vector of the trajectory path of the pilot hole, or compensate for any errors or deviations thereof from the pedicle axis is significantly reduced.

ANOTHER OBJECT of the present invention is to provide a method and apparatus that allows the surgeon to know the exact starting point of the pedicle, without any guessing, and create such starting point without burring.

A FURTHER OBJECT of the present invention is to provide a method and apparatus for use in spinal implant procedures that provides hemostasis and prevents over-penetration or plunging into the spinal cord at the initial stages on the pedicle screw insertion process.

YET ANOTHER OBJECT of the present invention is to provide a method and apparatus for use in spinal implant procedures which allows two surgeons to more easily conduct the instrumentation on the patient's spine at the same time, thereby dramatically reducing the time normally associated therewith.

These objects, as well as other objects and advantages will become apparent from the following disclosure.

### SUMMARY OF THE INVENTION

According to the present invention there is provided a pedicle punch for use in a pedicle screw insertion process. It is designed to be properly positioned against a vertebra pedicle, in as close alignment with the centrally located pedicle axis of the vertebra pedicle as possible, through minimal use of imaging technology, and once properly positioned to be deployed into the vertebrae pedicle quickly and effectively to create a pilot hole having (I) a diameter wide enough to accommodate the instrumentation of the next step in the pedicle screw insertion process; and (ii) a trajectory path vector that is as closely aligned to the pedicle axis of the vertebrae pedicle as possible.

The pedicle punch is formed of both a radiation translucent material and a radiation opaque material. It comprises a proximal end, a distal end, a shaft, a lip, a radiation opaque center core, and a radiation translucent outer layer. The proximal end acts as a handle for the pedicle punch. The shaft distally projects from the center of the proximal end and terminates to a sharp conical spike at the distal end. The lip outwardly projects from and is fixedly connected to the shaft, adjacent to the sharp conical spike and concentric thereto. The radiation opaque center core runs longitudinally along the entire length of the pedicle punch, through the proximal end, through the shaft and lip outwardly projecting therefrom, to the very tip of the distal end; and the radiation translucent outer layer concentrically surrounds and encases the radiation opaque center core, running along the entire length of the pedicle punch. However, it stops short a short distance from the very tip of the distal end to allow the radiation opaque center core to extend sufficiently beyond the radiation translucent outer layer and form the bottom of the sharp conical spike.

According to the present invention there is also provided a method for the use of the inventive pedicle punch comprising the steps of (a) accurately mapping the outer perimeter of the pedicle and the axis thereof; (b) placing the pedicle punch on the pedicle just mapped; c) moving and positioning the pedicle punch until the radiation opaque central core and the pedicle perimeter are “bulls' eyed”; (d) once pedicle perimeter and the central core are “bulls' eyed”, deploying the pedicle punch into the mapped vertebrae pedicle using an appropriate force to form a pilot hole having (I) a diameter wide enough to accommodate the instrumentation of the next step in the pedicle screw insertion process; and (ii) a trajectory path vector that is totally aligned with the pedicle axis of the vertebrae pedicle.

### BRIEF DESCRIPTION OF THE DRAWINGS

While the specification concludes with claims which particularly point out and distinctly claim the present invention, it is believed that the present invention will be better understood from the following detailed description taken in conjunction with the accompanying drawings in which like numerals represent identical elements and wherein:

FIG. 1 (prior art) is a diagram illustrating a vertebra with its pedicle section shown from all angles.;

FIG. 2 (prior art) is an artist's rendition of a pedicle conceptualized as a cylinder;

FIG. 3 is a perspective view of the preferred embodiment the pedicle punch according to the invention;

FIG. 4 is a longitudinal cross-sectional side view of an embodiment of the pedicle punch according to the invention showing the proximal handle, the shaft the sharp point at the distal end of the pedicle punch and the lip, wherein the lighter and darker shading indicating the two different materials comprising the punch.;

FIG. 5 is a top perspective of the preferred embodiment of the pedicle punch according to the invention;

FIG. 6 is a side view of the preferred embodiment of the pedicle punch according to the invention;

FIG. 7 is a cross-sectional view of the preferred embodiment of the pedicle punch according to the present invention of FIG. 6, taken along the line A-A highlighting the radiation opaque core and the radiation opaque protrusion on the distal side of the lip;

FIG. 8 is a bottom view of the preferred embodiment of the pedicle punch of FIG. 6 shown along the line B-B-;

FIG. 9 is a top view of the preferred embodiment of the pedicle punch of FIG. 6;

FIG. 10 is a top perspective of another embodiment of the pedicle punch of the present invention wherein the distal side of the lip bears no radiation opaque protrusions;

FIG. 11 is a side view of the pedicle punch of FIG. 10 showing serrations or barbs on the conical tip; and

FIG. 12 is a top view of the pedicle punch of FIG. 10.

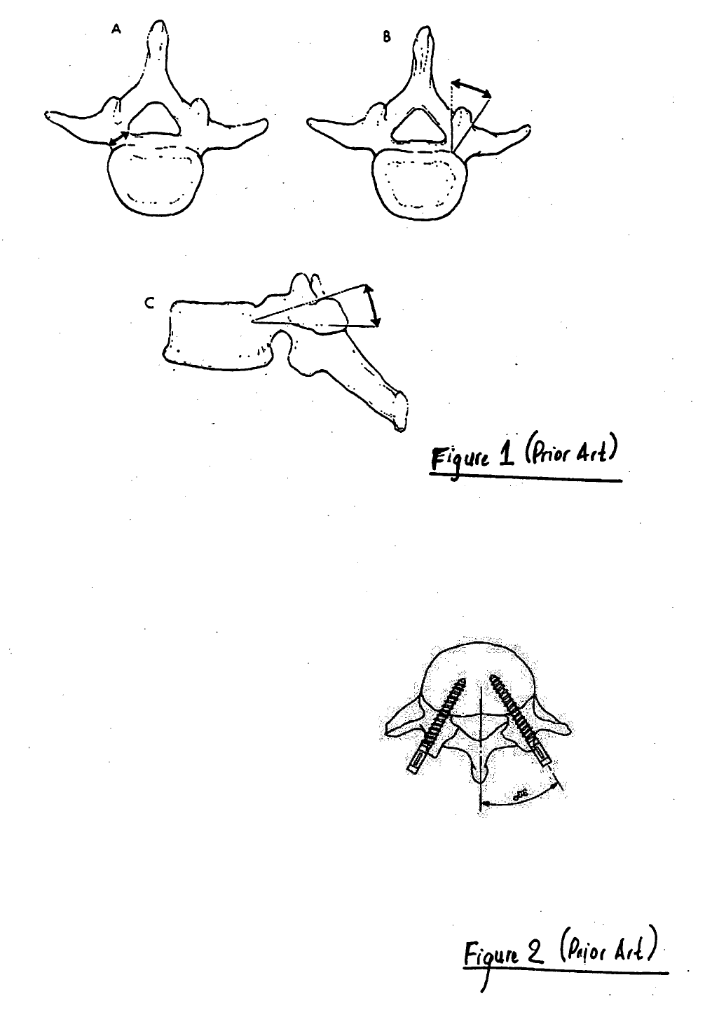

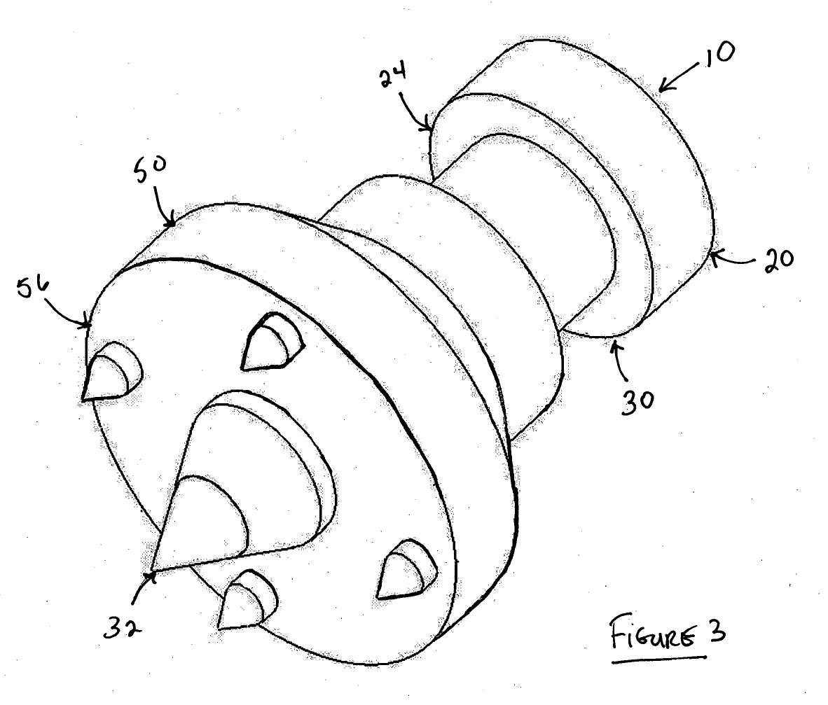

### LIST OF ELEMENTS AND THEIR RESPECTIVE IDENTIFYING NUMERALS

| NO | ELEMENT |
|---|---|
| 10 | Pedicle punch |
| 20 | Proximal end (handle) |
| 22 | proximal side of the proximal end 20 of the |
| | pedicle punch 10 |
| 24 | Distal side of the proximal end 20 of the |
| | pedicle punch 10 |
| 30 | Shaft |
| 32 | Sharp conical spike or pointy tip |
| 40 | Distal end of pedicle punch 10 |
| 50 | Outwardly protruding lip |
| 52 | Proximal side of the outwardly protruding lip |
| | 50 of the pedicle punch 10 |
| 54 | Distal side of the outwardly protruding lip 50 |
| | of the pedicle punch 10 |
| 56 | mini-protrusions |
| 60 | Radiation opaque or radiation dense center |
| | core of composite shaft 30 |
| 62 | Radiation translucent outer layer |
| 100 | Proximal circumferentially narrow end of the |
| | lip 50 of one of the preferred embodiments |
| 102 | Short outer edge of the proximal |
| | circumferentially narrow end 100 |
| 103 | outwardly directed edge of the lip 50 of one |
| | of the preferred embodiments |
| 110 | Distal circumferentially wide end of the lip |
| | 50 |
| 112 | Short outer edge of the distal |
| | circumferentially wide end 110. |
| 114 | Serrations, hooks, prongs or barbs |

### DETAILED DESCRIPTION OF THE INVENTION

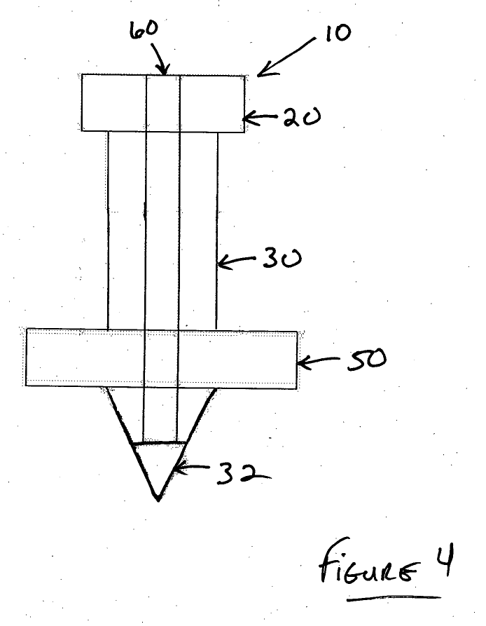

Referring more specifically to the drawings, FIGS. 3, 4, 5 and 10 generally depict the inventive pedicle punch at 10. The pedicle punch 10 is designed to replace whatever technology and tools currently being used to create pilot holes in pedicle vertebrae during spinal fusion surgical procedures, and more particular during the pedicle screw insertion process. It has been engineered to be properly positioned against the vertebrae pedicle, in as close alignment to the centrally located pedicle axis as possible, through minimal use of imaging technology, as for example x-rays, fluoroscopy, virtual fluoroscopy, etc., and once properly positioned to be deployed into the vertebrae pedicle quickly and effectively.

Once deployed into the pedicle, the pedicle punch 10 remains in position on the vertebrae pedicle until such time as the surgeons remove it to proceed to the next instrumentation step in the pedicle screw insertion process. When removed, the pedicle punch 10 leaves behind, on the vertebrae pedicle, a starter hole, also known as a pilot hole (hereinafter “a pilot hole”), having a diameter of 3-4 mm wide and a trajectory path vector that is as closely aligned to the pedicle axis of the vertebrae pedicle as possible, i.e., almost a perfect pilot hole. Thus, the surgeons will not have to engage in any further burring, or hole making, or checking with K-wires to determine whether in fact they have correctly identified the cental pedicle axis of the pedicle vertebrae before proceeding to the next step. Accordingly, not only will the inventive pedicle punch 10 help create the perfect pilot hole, but it will allow the surgeon to cut a tremendous amount of time off the pedicle screw insertion process, reduce a significant amount of radiation to which the surgical team and the patient are exposed to, and minimize the amount of bleeding during the procedure.

The pedicle punch 10 is formed of both radiation translucent and radiation dense material. The radiation translucent material could be any material, plastic, metal, or ceramic that allows the radiation of imaging technology used during the pedicle screw insertion processes, to travel right though the material. Furthermore, the radiation translucent material must also be very strong so that it can sustain the dynamic forces used on it during the deployment of the pedicle punch 10 into the vertebrae pedicle. In the preferred embodiment of the pedicle punch 10 the radiation translucent material used is plastic, not only for its ability to allow radiation through and its strength, but also for the low manufacturing costs associated therewith. The overall length of the pedicle punch 10 will be commensurate with the application.

Referring once again to FIGS. 3, 4, 5, 6, 7, 10 and 11, the pedicle punch 10 comprises a proximal end 20, a distal end 40, a shaft 30, an outwardly protruding lip 50, a radiation opaque or radiation dense center core 60 (hereinafter “the center core 60”) and a radiation translucent outer layer 62 encasing the center core 60, thereby being concentric thereto.

During use and deployment, the pedicle punch 10 will be held by the surgeons between their respective fingers, or any means that can hold the pedicle punch, e.g., a clamp, by the pedicle punch's proximal end 20. Preferably the means for holding the pedicle punch are radiation translucent so that they do not interfere with the use and application of the pedicle punch, as set forth herein below. Thus, the proximal end 20 can be disk shaped, or knob shaped, or any shape for that matter, so long as the shape is ergonomically designed to act as the handle of the pedicle punch 10. The proximal end 20 has a proximal side 22 and a distal side 24.

The shaft 30 is fixedly attached to and projects from the center of the distal side 24 of the proximal end 20 of the pedicle punch 10. It tapers to and ends in a sharp conical spike or pointy tip 32 at the distal end 40 of the pedicle punch 10.

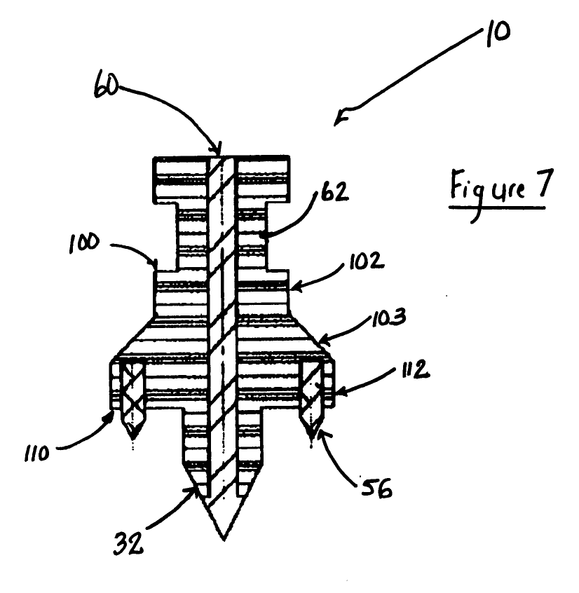

The outwardly protruding lip 50 is located right on the shaft 30. concentric thereto, and adjacent to the sharp conical spike or pointy tip 32, at the point where the shaft begins tapering to the sharp conical spike or pointy tip 32. It has a proximal side 52 and a distal side 54. Alternatively, as can be seen from FIGS. 6, 7 and 11, the outwardly protruding lip 50 is located right on the shaft 30, concentric thereto, adjacent to the sharp conical spike or pointy tip 32, but slightly above the point where the shaft begins tapering to sharp conical spike or pointy tip 32, whereby the shaft 30 extends a short length below the outwardly extending lip 50 before it begins tapering to form the sharp conical spike or pointy tip 32.

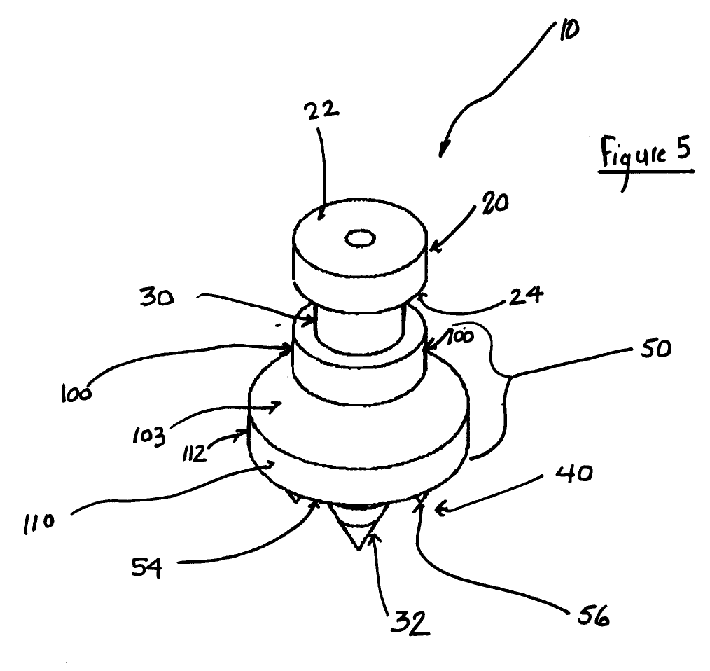

It has a proximal side 52 and a distal side 54. The thickness of the outwardly protruding lip 50 is not critical so long as it does not interfere with the proper location of the pedicle punch. However, it can range anywhere from 1-2mm in thickness. Its shape is generally square, as seen in FIG. 4, so that its proximate side forms a shelf with the shaft. Once the pedicle punch is deployed into the pedicle, the outwardly protruding lip 50 comes in direct contact with the pedicle and effectively seals any wound that is created by the pedicle punch as it is being deployed thereby imparting a hemostatic characteristic to the pedicle punch 10, as well. Consequently, the presence of the outwardly protruding lip 50 in combination with the fact that the punch is left in the wound, minimizes bleeding tremendously and significantly eliminates the use of thrombotic agents to stop the blood flow. Since excessive bleeding is no longer a critical problem, the present process might be expanded to be used with patients normally not recommended for spinal fusion surgery due to their inability to properly clot.

The center core 60 runs longitudinally along the entire length of the pedicle punch, through the proximal end 20, the center of the distal side 24 at the proximal end 20, and the very tip of the distal end 40. The radiation translucent outer layer 62 surrounds and encases the center core 60, thereby being concentric thereto. However, unlike the center core 60, it does not run the entire length of the pedicle punch. Instead, it stops short to allow the center core 60 to extend sufficiently beyond the distal edge of the outer layer 62 to form the bottom half of the sharp conical point or pointy tip 32 of the shaft 30, at the distal end 40 of the pedicle punch 10. Alternatively, if the radiation translucent outer layer 62 is even shorter in length, it can form the entire sharp conical point or pointy tip 32 of the shaft 30.

It is the radiation dense or radiation opaque center core 60 that allows the pedicle punch 10 to be placed concentrically with the vertebrae pedicle and in as close alignment to the pedicle axis as possible, to form a “bull's eye” with the pedicle upon the application of imaging technology, i.e. the center core 60 appears as the central solid disk and the pedicle appears as the outer circle, concentric to the central solid disk, normally associated with “bull's eye” configurations, and to ultimately allow the surgeon deploying the pedicle punch to create a pilot hole on the vertebrae pedicle that has a trajectory path vector in as close an alignment to the pedicle axis as possible. It should be noted that if the entire conical point or pointy tip 32 is formed completely of radiation opaque or radiation dense material, the pedicle punch's ability to form a “bull's eye” with the pedicle, upon the application of imaging technology will be severely compromised. Specifically, instead of the center core 60 together with the entire conical point 32 in conjunction with the pedicle circle appearing as a “bull's eye” under imaging technology, it will appear as a larger solid disk whose diameter will completely fill the pedicle circle, thereby completely masking the central axis of the pedicle.

In the preferred embodiment, metal is used to form the radiation opaque or radiation dense center core 60. Metal imparts great strength to the bottom end of the sharp conical spike or pointy tip 32 of the distal end 40 of the pedicle punch 10 and to the entire composite shaft 30. Such strength renders the pedicle punch 10 capable of not only creating the perfect pilot hole, as defined above, when deployed on the vertebrae pedicle, but of also sustaining the dynamic forces necessary to create the pilot hole without chipping, disintegrating, or otherwise compromising the integrity of the pedicle punch 10.

The specifications for the diameter of the entire sharp conical spike or pointy tip 32 comprising partly radiation translucent and partly radiation opaque material, from its widest point and tapering distally to form the pointy tip, will range so that when the pedicle punch 10 is deployed into the pedicle and thereafter removed therefrom, it leaves behind a pilot hole having dimensions of 3-4 mm. Obviously, if a larger pilot hole is desired then the diameter will be changed analogously. Similarly, the diameter at the most distal pointy tip end should be a diameter that will provide maximum penetrating power.

The pedicle punch 10 can also be equipped with tethering means, such as a suture, nylon string, or any other material that could be used by the surgeon to retrieve the pedicle from the patient.

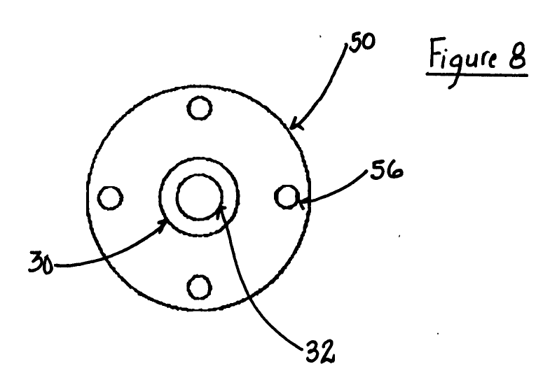

In a preferred embodiment of the inventive pedicle punch described hereinabove, the distal side 54 of the outwardly protruding lip 50 is provided with at least two teeth, knobs or mini protrusions 56 to stabilized and prevent toggling of the pedicle punch 10, once it is deployed in the vertebrae pedicle. The mini-protrusions 56 are spread as far away as possible from the center point of the distal side 54 of the outwardly protruding lip 50, to provide maximum balance. They are also made of radiation dense or radiation opaque mater, as shown in FIG. 7 so that they can enhance the “bull's eye” effect created by the center core 60 and the pedicle, under imaging technology.

The method for using the inventive pedicle punch 10 described above, comprises the following steps: (a) using an image generating apparatus, such a fluoroscope, to accurately map the pedicle and the axis thereof. The pedicle will be depicted as a circle on the apparatus screen, with the center point of the circle corresponding to the central axis of the pedicle; (b) placing the pedicle punch 10 on the vertebrae pedicle just mapped; c) manipulating, moving and positioning the pedicle punch 10 until such time that the radiation dense central core 60, which will appear as a solid dot on the screen of the image generating apparatus, is located right in the center of the circle corresponding to the pedicle just mapped, concentric to and in complete alignment with the mapped pedicle axis to form a “bulls' eye” (the solid center core 60 dot in the center and the pedicle circle concentrically aligned around said dot); (d) once the “bulls' eye” is achieved on the screen of the image generating apparatus, deploying the pedicle punch 10 into the mapped vertebrae pedicle using an appropriate force generating tool such as a surgical mallet; and (e) confirming the exact placement of the pedicle punch with the image making instrument and leaving the pedicle punch in place until needed to be removed. The surgeon is now ready for identification of the next pedicle and its axis and the steps are repeated until all of the pedicle punches are placed at all levels to be instrumented. Upon completion of the placement of the pedicle punches, the surgeon is ready to remove the pedicle punches one by one, and for each perfect pilot hole left behind on the vertebrae pedicle by the pedicle punch, to proceed to the next step of instrumentation in the pedicle screw insertion process.

In an alternate embodiment of the inventive pedicle punch described above, the proximal end 20 is provided with a groove for the purpose of providing the surgeon with a better grip on the pedicle punch 10.

In yet another embodiment of the invention described herein above, either the outer perimeter marking the outer edge of the proximal end 20, or the outer perimeter marking the outer edge of the outwardly protruding lip 50 is coated or otherwise provided with an opaque material, such that when the central core 60 is “bull's eyed” with the pedicle, the “bull's eye” comprises a center dot and two outwardly extending concentric circles, as opposed to one circle, concentric to the center dot, as described herein above.

In a further embodiment of the invention described herein above the pedicle punch's proximal end 20 is provided with cross hairs to better enhance the “bull's eye” effect created by the center core 60 with the pedicle under imaging technology.

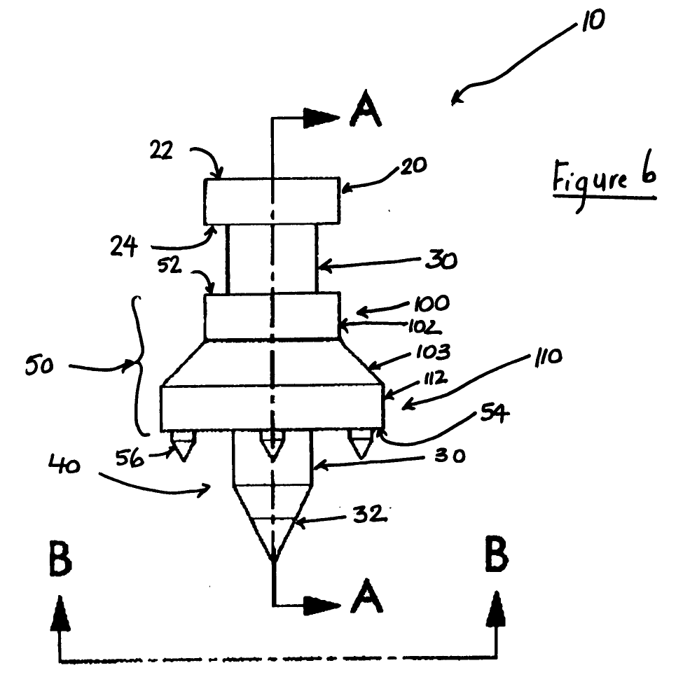

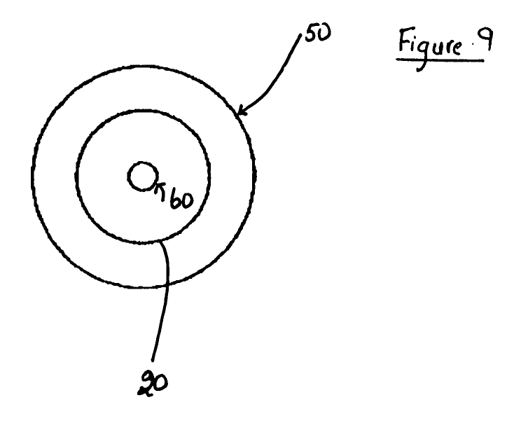

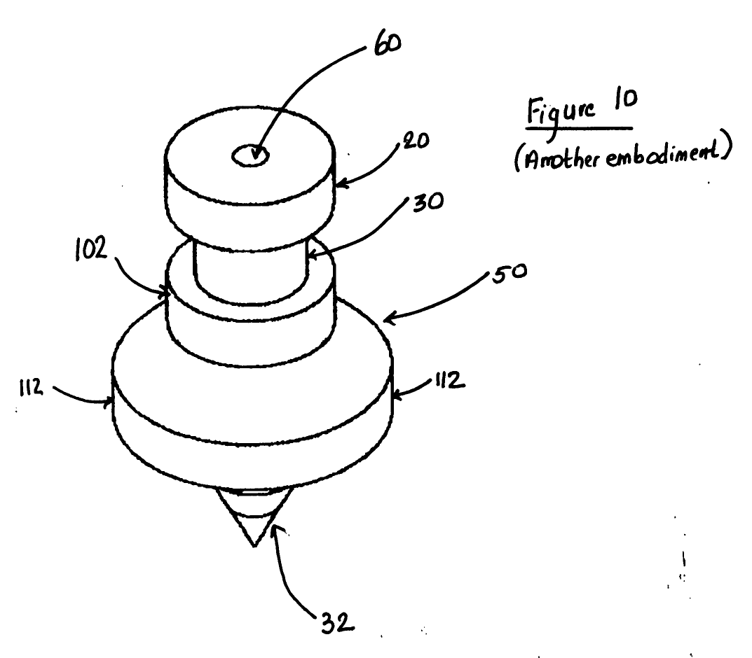

One of the preferred embodiments of the pedicle punch of the invention set forth herein above, is shown in FIG. 3 and in FIGS. 5-9. It comprises a proximal end 20, a shaft 30, an outwardly protruding lip 50, and a sharp conical spike or pointy tip 32. The center core 60 runs longitudinally along the entire length of the pedicle punch though the center of the proximal end, to the very tip of the distal end 40, extending sufficiently beyond the distal edge of the outer layer 62 to form only the bottom half of the sharp conical point or pointy tip 32 of the shaft 30, at the distal end 40 of the pedicle punch 10.

However, the lip 50 of the preferred embodiment is not square. Rather it has a composite, almost hat-like, shape having a proximal circumferentially narrow end 100 and a distal circumferentially wide end 110. The proximal circumferentially narrow end 100 has a relatively short outer edge 102, parallel to the central longitudinal axis of the center core 60, of the shaft 30, an upper end and a lower end. The distal circumferentially wide end 110 also has a relatively short outer edge 112, parallel to the central longitudinal axis of the center core 60, of the shaft 30, an upper end and a lower end. The lower end of the proximal outer edge 102 and the upper end of the distal outer edge 112 are connected with a third edge 103, directed outwardly and away from and angularly transverse to the central longitudinal center axis of the center core 60 to form a slightly sloped shoulder towards the distal circumferentially wide end 110 of the lip 50.

Furthermore, the lip 50 comprises four mini-protrusions 56 to maximize the stability of the deployed pedicle punch 10 by exerting an equal and opposite force to the force exerted on the pedicle by the sharp conical spike or pointy tip 32 both during deployment of the pedicle punch 10 and thereafter.

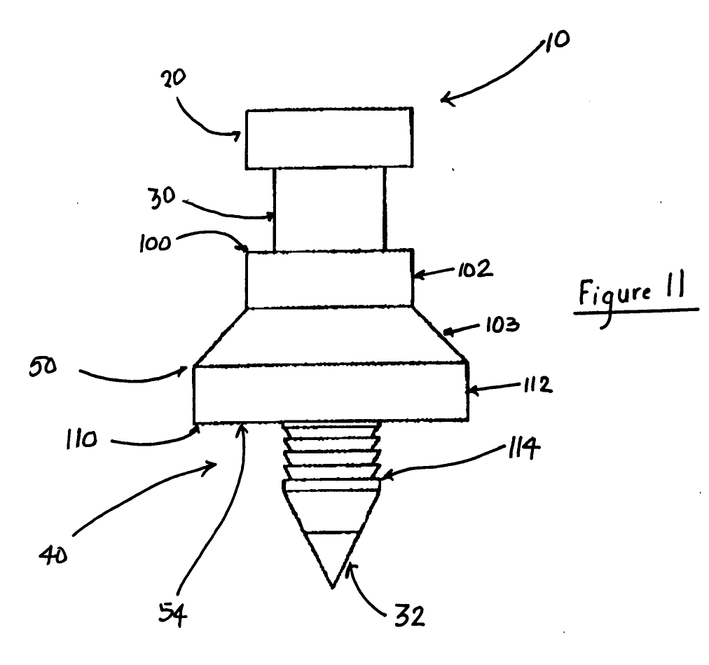

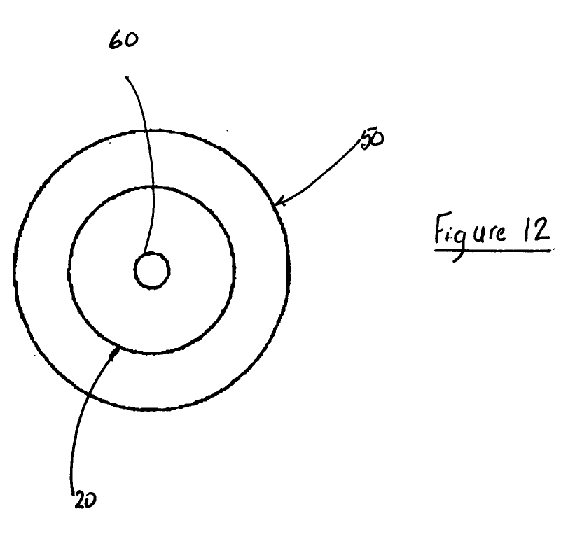

Another embodiment of pedicle punch of the invention set forth herein above, is shown in FIGS. 10-12. It too comprises a proximal end 20, a shaft 30, an outwardly protruding lip 50, a sharp conical spike or pointy tip 32, a center core 60 running longitudinally along the entire length of the pedicle punch, and a composite, almost hat-like-shaped lip having a proximal circumferentially narrow end 100 and a distal circumferentially wide end 110.

However, the composite-shaped lip 50 is totally devoid of any teeth, knobs or mini-protrusions whatsoever. Instead, the sharp conical spike or pointy tip 32 of the distal end 40 is provided with serrations, hooks, prongs, barbs or any other means 114 that will stabilize the pedicle punch 10 on the vertebrae pedicles, upon deployment thereon. Alternatively, the serrations, hooks, prongs, barbs or any other similar stabilizing means are located on the shaft below the distal side 54 of the lip 50, and adjacent to and immediately above the point on the shaft 30, at which that shaft begins tapering to and ending in a sharp conical spike or pointy tip 32 at the distal end 40 of the pedicle punch 10.

It is clear then from all of the above, that incorporating the pedicle punch 10 and the method of use thereof into spinal implant procedures and more particular into pedicle screw insertion processes accomplishes all of the invention's objectives as set forth herein above. It leads to the penetration of the pedicle with a probe and subsequent insertion of the pedicle screw quickly and accurately. Its ease of use significantly decreases the surgeons' learning curve, in that it allows the surgeon to know the exact starting point of the pedicle, without any guessing. It practically eliminates dural or neural injury during the creation of the pilot hole. It dramatically reduces the time normally associated with such procedures not only because it reduces the steps necessary for the creation of the perfect pilot hole, but because it can accommodate two surgeons working at the same time on one patient at a very rapid pace. Its minimum use of image making instruments and the speed at which the pedicle axis can be mapped and “bulls' eyed”, dramatically reduces the exposure of both the surgical team and the patient to radiation exposure and more particularly to fluoroscopic radiation exposure.

It also leads to a significant reduction of bleeding, partly because the burring of pilot holes is completely eliminated, thereby also eliminating the creation of pilot holes due to mistaken identification of pedicle axes; partly because the pedicle punch is left in the pilot holes until such time as the surgeons are ready to move to the next step in the pedicle screw insertion process; and partly because of the hemostatic effect of the pedicle punch due to its protruding lip. Accordingly, the inventive pedicle punch also minimizes the administration of thrombotic agents during the pedicle insertion process.

Finally, it unequivocally creates the perfect pilot hole, i.e., a pilot hole having (a) a diameter that is sized perfectly to accept the next instrument; and (b) a trajectory path vector in as complete alignment with the pedicle axis as possible, right from the very beginning of the pedicle penetration process.

While particular embodiments of the invention have been illustrated and described in detail herein, they are provided by way of illustration only and should not be construed to limit the invention. Those skilled in the art will readily recognize that various modifications and changes may be made to the present invention without departing from the spirit and intent of the present invention, as defined by the scope of the following claims.

What is claimed is:

1. A pedicle punch the pedicle punch used to establish pilot holes in vertebra pedicles when deployed thereon during pedicle screw insertion processes comprising:
a proximal end being ergonomically designed to act as a handle for the pedicle punch and a distal end;
a shaft distally projecting from the center of said proximal end and terminating to a sharp conical spike at said distal end;
a lip outwardly projecting from and fixedly connected to said shaft, adjacent to said sharp conical spike and concentric thereto;
a radiation opaque center core running longitudinally along the entire length of the pedicle punch, through said proximal end, through said shaft and lip outwardly projecting therefrom, to the very tip of said distal end; and
a radiation translucent outer layer concentrically surrounding and encasing said radiation opaque center core, running along the entire length of the pedicle punch but stopping short a short distance from the very tip of said distal end to allow said radiation opaque center core to extend sufficiently beyond the distal edge of said radiation translucent outer layer and form the bottom of said sharp conical spike of said shaft.

2. The pedicle punch according to claim 1, wherein said radiation translucent material is selected from the group of radiation translucent materials consisting of plastics, metals, and ceramics.

3. The pedicle punch according to claim 1, wherein said radiation translucent material is plastic.

4. The pedicle punch according to claim 1, wherein said radiation opaque material is metal.

5. The pedicle punch according to claim 1, wherein said lip further comprises at least two mini-protrusions to stabilize the pedicle punch as it is deployed into the vertebra pedicle.

6. The pedicle punch according to claim 5, wherein said mini-protrusions are radiation opaque.

9. The pedicle punch according to claim 1, wherein the lip further comprises four mini-protrusions to stabilize the pedicle punch as it is deployed into the vertebra pedicle.

10. The pedicle punch according to claim 9, wherein said four mini-protrusions are radiation opaque.

13. The pedicle punch according to claim 1 further comprising tethering means attached to said pedicle punch for pedicle punch easy retrieval.

14. The pedicle punch according to claim 1 wherein said sharp conical spike further comprises means for stabilizing the pedicle punch once deployed on the vertebra pedicle.

15. The pedicle punch according to claim 14, wherein said means for stabilizing said pedicle punch are barbs.

16. The pedicle punch according to claim 15, wherein said means for stabilizing said pedicle punch are serrations.

17. The pedicle punch according to claim 1, wherein said lip has a composite shape with a proximal circumferentially narrow end, a distal circumferentially wide end and an angled edge there between; said proximal circumferentially narrow end having a relatively short outer edge parallel to the central longitudinal axis of said radiation opaque center core with an upper end and a lower end; said distally circumferentially wide end having a relatively short outer edge parallel to the central longitudinal axis of said radiation opaque center core with an upper end and a lower end; said lower end of said proximal outer edge and said upper end of said distal outer edge being connected with said angled edge, such that said angled edge is angularly transverse to the central longitudinal center axis of said center core to form a slightly sloped shoulder towards said distal circumferentially wide end.

18. A pedicle punch used to establish pilot holes in vertebra pedicles when deployed thereon during pedicle screw insertion processes comprising:
a proximal end being ergonomically designed to act as a handle for the pedicle punch and a distal end;
a shaft distally projecting from the center of said proximal end and terminating to a sharp conical spike at said distal end, said sharp conical spike being provided with means for stabilizing the pedicle punch once deployed on the vertebra pedicle;
a lip outwardly projecting from and fixedly connected to said shaft, adjacent to said sharp conical spike and concentric thereto, said lip having a composite shape with a proximal circumferentially narrow end, a distal circumferentially wide end and an angled edge there between; said proximal circumferentially narrow end having a relatively short outer edge parallel to the central longitudinal axis of said radiation opaque center core with an upper end and a lower end; said distally circumferentially wide end having a relatively short outer edge parallel to the central longitudinal axis of said radiation opaque center core with an upper end and a lower end; said lower end of said proximal outer edge and said upper end of said distal outer edge being connected with said angled edge, such that said angled edge is angularly transverse to the central longitudinal center axis of said center core to form a slightly sloped shoulder towards said distal circumferentially wide end;
a radiation opaque center core running longitudinally along the entire length of the pedicle punch, through said proximal end, through said shaft and lip outwardly projecting therefrom, to the very tip of said distal end; and
a radiation translucent outer layer concentrically surrounding and encasing said radiation opaque center core, running along the entire length of the pedicle punch but stopping short a short distance from the very tip of said distal end to allow said radiation opaque center core to extend sufficiently beyond the distal edge of said radiation translucent outer layer and form the bottom of said sharp conical spike of said shaft.

19. A pedicle punch used to establish pilot holes in vertebra pedicles when deployed thereon during pedicle screw insertion processes comprising:
a proximal end being ergonomically designed to act as a handle for the pedicle punch and a distal end;
a shaft distally projecting from the center of said proximal end and terminating to a sharp conical spike at said distal end;
a lip outwardly projecting from and fixedly connected to said shaft, adjacent to said sharp conical spike and concentric thereto, said lip having a composite shape with a proximal circumferentially narrow end, a distal circumferentially wide end and an angled edge there between; said proximal circumferentially narrow end having a relatively short outer edge parallel to the central longitudinal axis of said radiation opaque center core with an upper end and a lower end; said distally circumferentially wide end having a relatively short outer edge parallel to the central longitudinal axis of said radiation opaque center core with an upper end and a lower end; said lower end of said proximal outer edge and said upper end of said distal outer edge being connected with said angled edge, such that said angled edge is angularly transverse to the central longitudinal center axis of said center core to form a slightly sloped shoulder towards said distal circumferentially wide end;
a radiation opaque center core running longitudinally along the entire length of the pedicle punch, through said proximal end, through said shaft and lip outwardly projecting therefrom, to the very tip of said distal end;
a radiation translucent outer layer concentrically surrounding and encasing said radiation opaque center core, running along the entire length of the pedicle punch but stopping short a short distance from the very tip of said distal end to allow said radiation opaque center core to extend sufficiently beyond the distal edge of said radiation translucent outer layer and form the bottom of said sharp conical spike of said shaft; and
at least two radiation opaque mini-protrusions to stabilize the pedicle punch as it is deployed into the vertebra pedicle, said at least two mini-protrusions fixedly attached to and distally extending from said lip.

20. A method for the creation of pilot holes in pedicle vertebrae during pedicle screw insertion processes, comprising the steps of:
(a) accurately mapping the outer perimeter of the pedicle and the axis thereof;
(b) placing a pedicle punch on the pedicle just mapped, said pedicle punch comprising a proximal end being ergonomically designed to act as a handle for the pedicle punch and a distal end, a shaft distally projecting from the center of said proximal end and terminating to a sharp conical spike at said distal end, a lip outwardly projecting from and fixedly connected to said shaft, adjacent to said sharp conical spike and concentric thereto, a radiation opaque center core running longitudinally along the entire length of the pedicle punch, through said proximal end, through said shaft and lip outwardly projecting therefrom, to the very tip of said distal end and a radiation translucent outer layer concentrically surrounding and encasing said radiation opaque center core, running along the entire length of the pedicle punch but stopping short a short distance from the very tip of said distal end to allow said radiation opaque center core to extend sufficiently beyond the distal edge of said radiation translucent outer layer and form the bottom of said sharp conical spike of said shaft;
c) moving and positioning said pedicle punch until said radiation opaque center core and the pedicle's perimeter are bulls' eyed;
(d) once said radiation opaque center core and said pedicle perimeter are bulls' eyed, deploying the pedicle punch into the mapped vertebrae pedicle using an appropriate force to form a pilot hole having a diameter wide enough to accommodate the instrumentation of the next step in the pedicle screw insertion process and a trajectory path vector that is as closely aligned to the pedicle axis of the vertebrae pedicle as possible.
